(19) United States
(12) Patent Application Publication
(10) Pub. No.: US 2025/0092344 A1
(43) Pub. Date: Mar. 20, 2025

(54) PROCESS AND SYSTEM FOR CONTINUOUS GAS FERMENTATION

(71) Applicant: LanzaTech, Inc., Skokie, IL (US)

(72) Inventors: Robert John Conrado, Washington, DC (US); Michael Emerson Martin, Chicago, IL (US); Jason Carl Bromley, Chicago, IL (US)

(21) Appl. No.: 18/468,083

(22) Filed: Sep. 15, 2023

(51) Int. Cl. C12M 1/107; C12M 1/00; C12P 7/06; C12P 7/16; C12P 7/24; C25B 1/55

(52) U.S. Cl. CPC C12M 21/04; C12M 47/10; C12P 7/06; C12P 7/16; C12P 7/24; C25B 1/55

(57) ABSTRACT

The disclosure is directed to a process and apparatus for continuous gas fermentation with reduced accumulation of co-products. The disclosure relates to the use of both a vacuum distillation unit and a membrane separator to provide efficient product recovery, cell retention, and water recycling to maximize production of the fermentation products. The disclosure provides for continuous product recovery at an effective rate and minimizes the accumulation of co-products in the fermentation broth while ensuring the retention and viability of the C1-fixing microorganisms.

### FIELD

This disclosure relates to systems and methods for continuous gas fermentation while minimizing the accumulation of co-products. In particular, the disclosure relates to recovering products from a fermentation broth, where the fermentation broth comprises viable microbial biomass, and where the recovery of the product along with viable microbial biomass is completed.

### BACKGROUND

Carbon monoxide (CO) and Carbon dioxide (CO₂) account for about 76% of global greenhouse gas emissions from human activities, with methane (16%), nitrous oxide (6%), and fluorinated gases (2%) accounting for the balance (the United States Environmental Protection Agency). The majority of CO and CO₂ comes from the burning of fossil fuels to produce energy, although industrial and forestry practices also emit greenhouse gases into the atmosphere. These greenhouse gases including CO and CO₂ can be utilized by catalytic processes, such as the Fischer-Tropsch process. However, extensive gas purification techniques are generally required in order to obtain a gas suitable for the Fischer-Tropsch process.

Gas fermentation has emerged as an advantageous alternative platform for the biological fixation of carbon and transformation or utilization of such carbon. In gas fermentation, carbon-rich gases such as carbon dioxide, carbon monoxide, or methane are converted by C1-fixing microorganisms into a wide range of products such as fuel, protein, and valuable chemical compounds. These products can be used by industries in the chemical, petrochemical, pharmaceutical, animal feed, environmental and agricultural sectors. The gas fermentation process may utilize a variety of feedstocks including those sourced from domestic, industrial, or agricultural waste, thereby reducing reliance on fossil sources of carbon and reducing the emission of greenhouse gases. Carbon that is already above-ground can be captured and recycled into new and useful products without further fossil carbon.

Products must be recovered from the gas fermentation broth. In batch-type operation, the fermentation process is stopped, and the product is recovered. Continuous processes are more effective commercially but bring a need to continuously recover the fermentation products at an effective rate to maintain the high production efficiency of the gas fermentation process. If the products accumulate in the continuous process to a level affecting the survival of the C1-fixing microorganisms, the fermentation process may have to be stopped and restarted resulting in costly downtime and regrowth of microorganisms. Other challenges in the production of desirable products from gas fermentation processes may include slow microbial growth, limited gas consumption, accumulation of unused nutrients, toxic co-products, or diversion of carbon substrates into undesired co-products. These challenges can be met by minimizing undesired washout of viable microorganisms and by controlling the liquid flow through the process to a target rate to continuously flush co-products from the system and avoid or minimize inhibitory and/or toxic effects of the co-products on the C1-fixing microorganisms. Selectively removing water and certain accumulated nutrients and co-products reduces the opportunity for the washout of viable microorganisms that might otherwise be removed through a high flow rate of the bleed stream that would be needed for overall liquid level control. Replenishment of which would require added expense.

Traditional distillation techniques for the separation and recovery of products and co-products usually operate at temperatures harmful to the C1-fixing microorganisms resulting in a loss of C1-fixing microorganisms. To overcome this loss, filtration methods alone have been employed. Often the filtration method employs one or more membranes. A drawback of using membrane filtration alone is that most membranes scale linearly with fermentation capacity because there is a required membrane surface area per volume of fermentation broth. Thus, membranes are typically more expensive to scale as compared to distillation. There is a need, in continuous gas fermentation processes, for an improved separation process for continuous product recovery and co-product removal that is not harmful to the microorganisms and that has increased efficiency. In other words, there is a need for a continuous and economic separation process and system that is capable of recovering desirable products at an effective rate along with reducing the accumulation of co-products and/or unused/accumulated nutrients such as salts, metals, vitamins all the while maximizing the viability of the C1-fixing microorganisms.

### SUMMARY

The disclosure provides a process for continuous gas fermentation with the benefit of reduced accumulation of co-products and/or unused nutrients. The process for continuous gas fermentation comprises generating, by gas fermentation of a gas stream in a bioreactor, a fermentation broth comprising gas, microbial biomass, at least one product, and at least one co-product; passing at least a portion of the fermentation broth to a separator and separating a gas stream from a liquid stream comprising the microbial biomass, the at least one co-product, and the at least one product; passing, serially or in parallel, at least a portion of the liquid stream to a vacuum distillation unit and a membrane separation unit; separating, in the vacuum distillation unit, a product enriched stream and a microbial biomass and co-product enriched stream; separating, in the membrane separation unit, a permeate stream enriched in water and co-product and a retentate stream enriched in microbial biomass; and recycling at least a portion of the retentate stream to the bioreactor.

The process may further comprise passing to the bioreactor, at least a portion of the microbial biomass and co-product enriched stream, at least a portion of the retentate, or both. The process may further comprise removing as a bleed stream, at least a portion at least a portion of the microbial biomass and co-product enriched stream, at least a portion of the retentate, or both. The process may further comprise passing at least a portion of the permeate stream to a wastewater treatment unit, generating a treated water stream, and passing at least a portion of the treated water stream to the bioreactor. The process may further comprise passing at least a portion of the gas stream to a gas treatment unit and removing at least one contaminant from the gas stream fermenting in the bioreactor. The microbial biomass and co-product enriched stream may comprise less than 0.2 wt. % product. The gas stream may comprise at least hydrogen and carbon monoxide wherein at least a portion of the carbon monoxide is generated by conversion of carbon dioxide to carbon monoxide. The conversion of carbon dioxide may comprise heating a gas stream comprising carbon-dioxide to a temperature in the range of about 400° C. to about 600° C., contacting with a conversion catalyst, and then cooling the resulting stream comprising carbon monoxide to a temperature in the range of about 35° C. to about 55° C. The conversion may employ reverse water gas shift conversion, CO₂ electrolysis conversion, thermo-catalytic conversion, electro-catalytic conversion, partial combustion conversion, plasma conversion, or any combination thereof. The carbon dioxide may be generated by steam reforming, electrolysis powered by a renewable power source, or both.

The disclosure also provides an apparatus for continuous gas fermentation comprising a bioreactor comprising a bioreactor inlet, at least one recycle inlet, and a fermentation broth outlet; a separator in fluid communication with the fermentation broth outlet, the separator comprising a liquid outlet and a gas outlet; a vacuum distillation unit and a membrane separator in fluid communication, serially or in parallel, with the separator liquid outlet; the vacuum distillation unit comprising an overhead outlet and a bottoms outlet; and the membrane separator comprising a permeate outlet and a retentate outlet, the retentate outlet in fluid communication with at least one of the bioreactor recycle inlets. The bottoms outlet may be further in fluid communication with at least one of the bioreactor recycle inlets. The apparatus may further comprise a wastewater treatment unit in fluid communication with the permeate outlet and at least one of the bioreactor recycle inlets. The apparatus may further comprise a gas treatment unit in fluid communication with the bioreactor inlet. The apparatus may further comprise a gas treatment unit in fluid communication with both the gas outlet and at least one of the bioreactor recycle inlets. The apparatus may further comprise, in fluid communication the bioreactor inlet, a CO₂ to CO conversion unit selected from a reverse water gas reaction unit, a CO₂ electrolysis unit, a thermo-catalytic conversion unit, an electro-catalytic conversion unit, a partial combustion unit, a plasma conversion unit or any combination thereof. The apparatus may further comprise a bleed conduit in fluid communication with the liquid outlet, retentate outlet, or both.

The vacuum distillation unit is designed to transfer viable microbial biomass back to the bioreactor. The microbial biomass is contained in the product depleted stream. At least a portion of the product depleted stream is recycled to the bioreactor. The product depleted stream comprises less than 0.2 wt. % product relative to the fermentation broth to prevent, or at least mitigate, accumulation of the product in the fermentation broth. To effectively remove the product from the fermentation broth, the vacuum distillation operates at a pressure below atmospheric pressure and at a temperature range capable of removing product, while ensuring the viability of the microorganisms. In an embodiment, the vacuum distillation unit may be operated at a pressure in the range of about 40 mbar(a) to about 100 mbar(a) and at a temperature in the range of about 35° C. to about 50° C.

In an embodiment, at least a portion of the product depleted stream is passed to a membrane separator and separated into a permeate stream enriched in water and at least one co-product and a retentate stream enriched in microbial biomass and water. The retentate stream is recycled to the bioreactor. At least a portion of the product depleted stream is removed as a bleed stream before passing it to the membrane separator. At least a portion of the permeate stream may be passed to a wastewater treatment unit to obtain a treated water stream. At least a portion of the treated water stream may be passed to the bioreactor.

The product may be at least one selected from ethanol, acetate, butanol, butyrate, 2,3-butanediol, lactate, butene, butadiene, methyl ethyl ketone (2-butanone), ethylene, acetone, isopropanol, lipids, 3-hydroxypropionate (3-HP), terpenes, terpenoids, isoprene, fatty acids, 2-butanol, 1,2-propanediol, 1 propanol, chorismate-derived products, 3 hydroxybutyrate, and 1,3-butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, monoethylene glycol, 2-phenylethanol, or any combination thereof. In certain instances, one or more by-products are not separated from the fermentation broth and are returned to the bioreactor in the product depleted stream.

The process further comprises generation of the fermentation broth by gas fermentation, comprising obtaining a first gaseous stream comprising H₂ and a second gaseous stream comprising CO₂. At least a portion of the first gaseous stream is passed to the bioreactor. The second gaseous stream is passed to a CO₂ to CO conversion system to obtain a gaseous feed stream enriched in CO. In an embodiment, the second gaseous stream may be heated to a temperature in the range of about 400° C. to about 600° C. before passing to the CO₂ to CO conversion system. The gaseous feed stream is passed to the bioreactor. The bioreactor is operated under conditions for the fermentation of a C1-containing gas. In certain instances, the temperature of the gaseous feed stream may be elevated that needs to be cooled before being passed to the bioreactor as higher temperatures may result in a decrease in microorganism viability. The gaseous feed stream may be cooled to a temperature in the range of about 35° C. to about 55° C. before passing to the bioreactor. The second gaseous stream may be combined with at least a portion of the first gaseous stream to obtain a combined gas stream. The combined gas stream may be passed to the CO₂ to CO conversion system. The first gaseous stream is generated by steam reforming, electrolysis, or both, wherein the electrolysis is powered by a renewable power source.

### BRIEF DESCRIPTION OF THE DRAWINGS

The figures have been simplified by the deletion of a large number of apparatuses customarily employed in a process of this nature, such as vessel internals, temperature and pressure control systems, flow control valves, recycle pumps, and the like. which are not specifically required to illustrate the performance of the invention. Furthermore, the illustration of the process of this invention in the embodiment of a specific drawing is not intended to limit the disclosure to specific embodiments. Some embodiments may be described by reference to the process configuration shown in the figure, which relates to both apparatus and processes to carry out the disclosure. Any reference to a process step includes reference to an apparatus unit or equipment that is suitable to carry out the step, and vice-versa.

FIG. 1a is a schematic flow diagram showing the continuous process and apparatus for separating products, separating and recycling microbial biomass, and separating and removing co-products comprising vacuum distillation unit, and membrane separator in series, in accordance with one embodiment of the disclosure.

FIG. 1b is a schematic flow diagram showing the continuous process and apparatus for separating products, separating and recycling microbial biomass, and separating and removing co-products comprising vacuum distillation unit, and membrane separator in parallel, in accordance with other embodiment of the disclosure.

FIG. 2a shows a metabolite plot resulting from the use of vacuum distillation in a gas fermentation process.

FIG. 2b and FIG. 2c show metabolite plots resulting from the use of vacuum distillation and membrane separator in a gas fermentation process.

### DETAILED DESCRIPTION

The disclosure provides a process and apparatus for separating and recovering products, separating and recycling microbial biomass, and also separating and removing co-products. The apparatus comprises at least a bioreactor containing a biocatalyst, vacuum distillation unit, and membrane separator. In addition, at least one product may be effectively recovered from a fermentation broth, containing viable microbial biomass, while preserving the viability of the microbial biomass using a combination of separation techniques and controlling the liquid level within the system.

The stream flowing into a gas fermentation bioreactor may be termed as “gas fermentation feedstock” or “feedstock” and should be understood to encompass any material (solid, liquid, or gas) or stream that can provide a substrate and/or C1-carbon source to a bioreactor either directly or after processing of the feedstock. The bioreactor includes a culture of one or more C1-fixing microorganisms that have the ability to produce one or more products from a C1-carbon source. “C1” refers to a one-carbon molecule, for example, CO, CO₂, CH₄, or CH₃OH. “C1-carbon source” refers to a one-carbon molecule that serves as a partial or sole carbon source for the microorganism. For example, a C1-carbon source may comprise one or more of CO, CO₂, CH₄, CH₃OH, or CH₂O₂. In an embodiment, the C1-carbon source comprises one or both of CO and CO₂. “Substrate” refers to a carbon and/or energy source for the microorganism. Typically, the substrate is gaseous and comprises a C1-carbon source, for example, CO, CO₂, and/or CH₄. The substrate may further comprise other non-carbon components, such as H₂, N₂, and/or electrons.

The substrate and/or C1-carbon source may be a gas obtained as a by-product of an industrial process or from another source, such as combustion engine exhaust fumes, biogas, landfill gas, direct air capture, flaring, or from electrolysis. The substrate and/or C1-carbon source may be syngas generated by pyrolysis, torrefaction, or gasification. In other words, carbon in solid or liquid materials may be recycled by pyrolysis, torrefaction, or gasification to generate syngas which is used as the substrate and/or C1-carbon source in gas fermentation. The substrate and/or C1-carbon source may be natural gas. The substrate and/or C1-carbon source carbon dioxide from conventional and unconventional gas production. The substrate and/or C1-carbon source may be a gas comprising methane. Gas fermentation processes are flexible and any of these substrates and/or C1-carbon sources may be employed.

In certain embodiments, the industrial process source of the substrate and/or C1 carbon source is selected from ferrous metal products manufacturing, such as steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, the industrial processing of geological reservoirs, processing fossil resources such as natural gas coal and oil, landfill operations, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top gas, and residual gas from smelting iron. Other general examples include flue gas from fired boilers and fired heaters, such as natural gas, oil, or coal-fired boilers or heaters, and gas turbine exhaust. Another example is the flaring of compounds such as at oil and gas production sites. In these embodiments, the substrate and/or C1-carbon source may be captured from the industrial process before it is emitted into the atmosphere, using any known method.

The substrate and/or C1-carbon source may be a synthesis gas known as syngas, which may be obtained from reforming, partial oxidation, plasma, or gasification processes. Examples of gasification processes include the gasification of coal, gasification of refinery residues, gasification of petroleum coke, gasification of biomass, gasification of lignocellulosic material, gasification of wood, gasification of black liquor, gasification of municipal solid waste, gasification of municipal liquid waste, gasification of industrial solid waste, gasification of industrial liquid waste, gasification of refuse-derived fuel, gasification of sewerage, gasification of sewerage sludge, gasification of sludge from wastewater treatment, gasification of landfill gas, gasification of biogas such as when biogas is added to enhance gasification of another material. Examples of reforming processes include steam methane reforming, steam naphtha reforming, reforming of natural gas, reforming of biogas, reforming of landfill gas, reforming of coke oven gas, reforming of pyrolysis off-gas, reforming of ethylene production off-gas, naphtha reforming, and dry methane reforming. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse-derived fuel, and fibers in many day-to-day products such as shoes, apparel, and textiles. Municipal solid waste may be simply landfill-type waste and may be sorted or unsorted. Examples of biomass may include lignocellulosic material and microbial biomass. Lignocellulosic material may include agriculture by-products, forest by-products, and some industrial by-products.

Biomass may be created as by-products of “nature-based solutions” (NbS) and thus nature-based solutions may provide feedstock to the gas fermentation process. Nature-based solutions are articulated by the European Commission as solutions inspired and supported by nature, which are cost-effective, simultaneously provide environmental, social, and economic benefits and help build resilience. Such solutions bring more, and more diverse, nature and natural features and processes into cities, landscapes, and seascapes, through locally adapted, resource-efficient, and systemic interventions. Nature-based solutions must benefit biodiversity and support the delivery of a range of ecosystem services. Through the use of NbS healthy, resilient, and diverse ecosystems (whether natural, managed, or newly created) can provide solutions for the benefit of both societies and overall biodiversity. Examples of nature-based solutions include natural climate solutions (conservation, restoration, and improved land management that increase carbon storage or avoid greenhouse gas emissions in landscapes and wetlands across the globe), halting biodiversity loss, socio-economic impact efforts, habitat restoration, and health and wellness efforts with respect to air and water. Biomass produced through nature-based solutions may be used as feedstock to gas fermentation processes.

The substrate and/or C1-carbon source may be a gas stream comprising methane. Such a methane-containing gas may be obtained from fossil methane emissions such as during fracking, wastewater treatment, livestock, agriculture, and municipal solid waste landfills. It is also envisioned that the methane may be burned to produce electricity or heat and the C1 by-products may be used as the substrate or carbon source. The substrate and/or C1-carbon source may be a gas stream comprising natural gas.

An optional step of a gasification process in the overall gas fermentation process greatly increases suitable feedstocks for the overall gas fermentation process as compared to gaseous feedstocks alone. The gasification process converts organic and/or fossil fuel-based “carbonaceous materials” into carbon monoxide (CO), hydrogen (H₂), and carbon dioxide (CO₂). Further, incentives achieved may extend beyond items such as carbon credits, and into the natural-based solutions space. The gasification process may include various technologies including but not limited to, counter-current fixed bed gasifiers, co-current fixed bed gasifiers, fluidized bed reactors, entrained flow gasifiers, and plasma gasifiers. The gasification process may utilize any feed, which can produce a syngas stream. In an embodiment, the gas fermentation feedstock of the present disclosure is generated by the gasification process. The term “gasification process” encompasses the gasifier itself along with unit operations associated with gasification, including the heating source for the gasifier and syngas quench processes.

As used herein, “carbonaceous material” refers to carbon-rich materials such as coal and petrochemicals. However, carbonaceous material includes any carbon material whether in a solid, liquid, gaseous, or plasma state. Among the large number of items that can be considered carbonaceous materials, the present disclosure contemplates: carbonaceous material, carbonaceous liquid product, carbonaceous industrial liquid recycle, carbonaceous municipal solid waste (MSW), carbonaceous municipal waste, carbonaceous agricultural material, carbonaceous forestry material, carbonaceous wood waste, carbonaceous building material, carbonaceous plant material, carbonaceous industrial waste, carbonaceous fermentation waste, carbonaceous petrochemical co-products, carbonaceous alcohol production co-products, semi-anthracite, tires, plastics, waste plastics, coke oven tar, soft fiber (fibersoft), lignin, black liquor, polymers, waste polymers, polyethylene terephthalate (PETA), Polystyrene (PS), sewage sludge, animal waste, crop residue, energy crops, forestry processing residue, wood processing residue, livestock manure, poultry manure, food processing residue, fermentation process waste, ethanol co-products, distillers grains, waste microorganisms, or combinations thereof.

In an embodiment, gasifying a gasification feedstock to produce syngas may further comprise: (i) introducing oxidant and gasifier feedstock comprising waste material to gasifier; and (ii) processing gasifier gaseous product. Processing gasifier gaseous product may comprise one or more of: removing heat from gasifier gaseous product; removing particulate matter from gasifier gaseous product and cleaning up gasifier gaseous product. In another embodiment, the gasification feedstock may be municipal/industrial solid waste, agricultural waste, microbial biomass, or any combination thereof. The gasification feedstock may be dried in a dryer and then gasified to produce a gas fermentation feedstock. At least a portion of the gas fermentation feedstock is passed to a fermentation process to produce one or more products and possibly at least one co-product. In some embodiments, the microbial biomass produced from the fermentation process may be passed to the gasification operation as a feedstock for gasification.

Syngas produced by gasification techniques involves cost. Therefore, it is advantageous to use syngas efficiently both in the fermentation process to make higher-value products and in conserving the syngas values in any clean-up operation. The financial feasibility of any gas fermentation process, especially to produce commodity chemicals such as ethanol and acetic acid, is dependent on the efficiency of conversion of the feedstock to the sought products and the energy costs to affect the conversion in addition to capital costs.

Gas fermenting microorganisms or C1-fixing microorganisms can utilize a wide range of feedstocks including syngas generated from gasified organic matter of any sort (i.e., municipal solid waste, industrial waste, biomass, and agricultural waste residues) or industrial off-gases (i.e., from steel mills or other processing plants). Different feedstocks may contain impurities in the substrates due to the source of the feedstock, processing of the feedstocks, and variables and trace elements present in the feedstocks. Some impurities can affect the downstream conversion performance of gas-fermenting microorganisms. Therefore, the gas fermentation feedstock may be treated in a gas treatment unit to remove impurities that are fermentation inhibitors. This process is also known as gas clean-up. In an embodiment, the gas treatment system may comprise a hydrolysis module, an acid gas removal module, a deoxygenation module, or any combination thereof. In another embodiment, a single module is used for all functions to remove impurities that are fermentation inhibitors. At least one gas fermentation inhibitor present in the feedstock may be removed and/or converted by the hydrolysis function. At least one of the impurities selected from carbon dioxide (CO₂), hydrogen sulfide (H₂S), and hydrogen cyanide (HCN) may be removed and/or converted by the acid gas removal function. At least one of the impurities selected from oxygen (O₂) and/or acetylene (C₂H₂) may be removed and/or converted by the deoxygenation function. Suitable processes for removing gas fermentation inhibitors from gas fermentation feedstocks to provide a suitable gas for a downstream fermentation process may be found in WO 2019/157519, and U.S. Pat. No. 11,441,116.

In the embodiment where a single gas treatment module is employed, at least one gas fermentation inhibitor present in the gas fermentation feedstock may be removed and/or converted in the gas treatment unit through contact with one or more specialized catalysts. For example, a specialized catalyst may be used to reduce oxygen to less than 100 ppm, acetylene to less than 1 ppm, and hydrogen cyanide to less than 1 ppm. Examples of such specialized catalysts comprise reduced copper metal on a high surface area catalyst such as silica, alumina, titania, ceria, lanthana, silica-alumina, carbon, or many other materials known to those skilled in the art. In certain instances, one specialized catalyst may be is copper (I) supported on alumina. In certain instances, the specialized catalyst comprises sulfided copper (I) supported on alumina, such that it is tolerant to sulfur. In certain instances, the specialized catalyst comprises copper (II) supported on alumina. In certain instances, the specialized catalyst comprises sulfided copper (II) supported on alumina, such that it is tolerant to sulfur. When treating feedstock with high sulfur content, the specialized catalyst may comprise sulfided copper supported on alumina.

The types or ratios of components in the gasification feedstock being gasified may change and result in changes in gasifier performance and syngas composition, including the type and amount of fermentation inhibitors in the resulting syngas. Thus, variabilities in types and concentrations of components adverse to the fermentation process, such as hydrogen cyanide, nitric oxide, acetylene, and ethylene, may occur. It is therefore advantageous for gas treatment or clean-up operations to have sufficient capacity to handle peak amounts of different impurities.

Depending on the desired composition of syngas, the gasification process may comprise one or more gas treatment systems or clean-up units to remove impurities or non-syngas components from the syngas produced by gasification. Suitable gas treatment or gas clean-up units include any of those known in the art, such as for example, acid gas removal units may be used to remove extraneous carbon dioxide. Such removed carbon dioxide may, depending on plant locations, be sold for purposes such as enhanced oil recovery operations.

A “C1-fixing microorganism” is a microorganism that has the ability to produce one or more products from a C1-carbon source. Typically, the microorganism contained in the bioreactor is a C1-fixing bacterium. A “microorganism” or “biocatalyst” is a microscopic organism, especially a bacterium, archaea, virus, or fungus. The microorganism is typically a bacterium. As used herein, recitation of “microbial biomass” should be taken to encompass “bacterium”.

The microorganisms contained in the bioreactor may be modified from a naturally occurring microorganism. A “parental microorganism” is a microorganism used to generate a microorganism. The parental microorganism may be a naturally occurring microorganism, known as a wild-type microorganism or a microorganism that has been previously modified, known as a mutant or recombinant microorganism. The microorganism contained in the bioreactor may be modified to express or overexpress one or more enzymes that were not expressed or overexpressed in the parental microorganism. Similarly, the microorganism contained in the bioreactor may be modified to contain one or more genes that were not contained by the parental microorganism. The microorganism contained in the bioreactor may also be modified to not express or to express lower amounts of one or more enzymes that were expressed in the parental microorganism. In one embodiment, the parental microorganism is Clostridium autoethanogenum, Clostridium ljungdahlii, or Clostridium ragsdalei. In an embodiment, the parental microorganism is Clostridium autoethanogenum LZ1561, which was deposited on Jun. 7, 2010, with Deutsche Sammlung von Mikroorganismen und Zellkulturen GmbH (DSMZ) located at Inhoffenstraße 7B, D-38124 Braunschweig, Germany on Jun. 7, 2010, under the terms of the Budapest Treaty and accorded accession number DSM23693. This strain is described in International Patent Publication No. WO 2012/015317.

The microorganism contained in the bioreactor may be cultured with the feedstock and produce one or more gas fermentation products. For instance, the microorganism may produce or may be engineered to produce ethanol (WO 2007/117157, U.S. Pat. No. 7,972,824), acetate (WO 2007/117157, U.S. Pat. No. 7,972,824), 1-butanol (WO 2008/115080, U.S. Pat. No. 8,293,509, WO 2012/053905, U.S. Pat. No. 9,359,611 and WO 2017/066498, U.S. Pat. No. 9,738,875), butyrate (WO 2008/115080, U.S. Pat. No. 8,293,509), 2,3-butanediol (WO 2009/151342, U.S. Pat. No. 8,658,408 and WO 2016/094334, U.S. Pat. No. 10,590,406), lactate (WO 2011/112103, U.S. Pat. No. 8,900,836), butene (WO 2012/024522, US2012/045,807), butadiene (WO 2012/024522, US 2012/045,807), methyl ethyl ketone (2-butanone) (WO 2012/024522, US 2012/045,807 and WO 2013/185123, U.S. Pat. No. 9,890,384), ethanol which is then converted to ethylene (WO 2012/026833, US 2013/157,322), acetone (WO 2012/115527, U.S. Pat. No. 9,410,130), isopropanol (WO 2012/115527 U.S. Pat. No. 9,410,130), lipids (WO 2013/036147 U.S. Pat. No. 9,068,202), 3-hydroxypropionate (3-HP) (WO 2013/180581, U.S. Pat. No. 9,994,878), terpenes, including isoprene (WO 2013/180584, U.S. Pat. No. 10,913,958), fatty acids (WO 2013/191567 U.S. Pat. No. 9,347,076), 2-butanol (WO 2013/185123 U.S. Pat. No. 9,890,384), 1,2-propanediol (WO 2014/036152, U.S. Pat. No. 9,284,564), 1-propanol (WO 2014/0369152, U.S. Pat. No. 9,284,564), 1 hexanol (WO 2017/066498, U.S. Pat. No. 9,738,875), 1 octanol (WO 2017/066498, U.S. Pat. No. 9,738,875), chorismate-derived products (WO 2016/191625, U.S. Pat. No. 10,174,303), 3-hydroxybutyrate (WO 2017/066498, U.S. Pat. No. 9,738,875), 1,3-butanediol (WO 2017/066498, U.S. Pat. No. 9,738,875), 2-hydroxyisobutyrate or 2-hydroxyisobutyric acid (WO 2017/066498, U.S. Pat. No. 9,738,875), isobutylene (WO 2017/066498, U.S. Pat. No. 9,738,875), adipic acid (WO 2017/066498, U.S. Pat. No. 9,738,875), 1,3-hexanediol (WO 2017/066498, U.S. Pat. No. 9,738,875), 3-methyl-2-butanol (WO 2017/066498, U.S. Pat. No. 9,738,875), 2-buten-1-ol (WO 2017/066498, U.S. Pat. No. 9,738,875), isovalerate (WO 2017/066498, U.S. Pat. No. 9,738,875), isoamyl alcohol (WO 2017/066498, U.S. Pat. No. 9,738,875), and/or monoethylene glycol (WO 2019/126400, US 2019/0185,888) in addition to 2-phenylethanol (WO 2021/188190, US2021/0292732).

The microorganism contained in the bioreactor may be engineered to produce products at a certain selectivity or at a minimum selectivity. In one embodiment, the target product accounts for at least 10% of all fermentation products produced by the microorganism, such that the microorganism has a selectivity for the target product of at least 10%. In another embodiment, the target product accounts for at least 30% of all fermentation products produced by the microorganism, such that the microorganism has a selectivity for the target product of at least 30%.

The culture is generally maintained in an aqueous culture medium that contains nutrients, vitamins, and/or minerals sufficient to permit the growth of the microorganism. The aqueous culture medium may be an anaerobic microbial growth medium. The culture/fermentation should desirably be carried out under appropriate conditions for the production of the target product. Typically, the culture/fermentation is performed under anaerobic conditions. Reaction conditions to consider include pressure or partial pressure, temperature, gas flow rate, liquid flow rate, media pH, media redox potential, agitation rate when employing a continuous stirred tank reactor, inoculum level, maximum gas substrate concentrations so that gas in the liquid phase does not become limiting, and maximum product concentrations to avoid product inhibition. In particular, the rate of introduction of the substrate may be controlled so that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, the culture/fermentation may be conducted at pressures higher than atmospheric pressure. Also, since a given gas conversion rate is, in part, a function of the substrate retention time and retention time dictates the required volume of a bioreactor, the use of pressurized systems can reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used.

The bioreactor includes a fermentation device consisting of one or more units and/or towers or piping arrangements. The bioreactor may be a continuous stirred tank reactor (CSTR), immobilized cell recycles (ICR), trickle bed reactor (TBR), bubble column, gas lift fermenter, static mixer, a circulated loop reactor, a membrane reactor, such as a hollow fibre membrane bioreactor (HFM BR) or other unit or other devices suitable for gas-liquid contact. The reactor may be adapted to receive a gaseous substrate comprising CO and/or CO₂, or H₂ or mixtures thereof. The reactor may comprise multiple reactors or stages, either in parallel or in series. For example, the reactor may comprise a first growth reactor in which the bacteria are cultured and a second fermentation reactor, to which fermentation broth from the growth reactor may be fed and in which most of the fermentation products may be produced. The fermentation process may be described as either “batch” or “continuous”. “Batch fermentation” is used to describe a fermentation process where the bioreactor is filled with raw material, i.e., the carbon source, along with microorganisms, where the products remain in the bioreactor until fermentation is completed. In a “batch” process, after fermentation is completed, the products are extracted, and the bioreactor is cleaned before the next “batch” is started. “Continuous fermentation” is used to describe a fermentation process where the fermentation process is extended for longer periods of time, and product and/or metabolite is removed during fermentation.

The fermentation broth generated from the bioreactor encompasses a mixture of components including the nutrient media, the culture of one or more microorganisms, and one or more products. “Nutrient media” or “nutrient medium” is used to describe culture growth media. Generally, this term refers to a media containing nutrients and other components appropriate for the growth of the microbial culture. The term “nutrient” includes any substance that may be utilized in a metabolic pathway of a microorganism. Exemplary nutrients include potassium, B vitamins, trace metals, and amino acids.

In an embodiment, a pump or a compressor may be provided upstream of the bioreactor so that the pressure within the bioreactor is increased. A pump or compressor may be provided to facilitate the delivery of the streams to particular stages. Furthermore, a compressor can be used to increase the pressure of gas provided to one or more stages, for example, the bioreactor. The pressure within a bioreactor can affect the solubility of gaseous substrate in the nutrient media thus increasing the efficiency of the fermentation reaction performed therein. Thus, the pressure can be adjusted to improve the efficiency of the fermentation. While the disclosure broadly describes any type of stream that may be moved through or around the system(s) by any known transfer means, in certain embodiments, the substrate and/or exhaust streams are gaseous.

The fermentation broth generated from the bioreactor may comprise waste gases generated as byproducts in gas fermentation, inert gases, and/or unused substrate gas, which may need to be removed from the broth. Typical gasses to be separated from a gas fermentation broth may include CO₂, CO, and/or H₂. The broth effluent further contains microbial biomass and liquid nutrient solution, in addition to the target product or products, and other metabolites or co-products. Waste gases not removed from the gas fermentation broth are recycled to the bioreactor with the recycled broth. Recycled inert gases and/or CO₂ dilute or restrict the amount of new gas substrate that is capable of being absorbed into the recycled broth; such new gas substrate is needed to be available for fermentation by the microbial biomass. Further, to maximize fermentation by the microbial biomass, smaller substrate bubbles should be generated by the reactor system. Smaller bubbles may be created by increasing system superficial gas velocities, see U.S. application Ser. No. 17/453,476 filed Nov. 3, 2021. Residual inert gasses and/or CO₂ not removed from the system and instead recycled in the broth may expand within the reactor restricting superficial gas velocities and limiting the creation of smaller bubbles such as fine bubbles. Therefore, at least a portion of the fermentation broth is passed to a separator. The separator operates to separate a gas stream from the fermentation broth. In an embodiment, the separator is a gas-liquid separator or a degasser.

A separated gas stream is removed from the separator. The separated gas stream comprising the dissolved gas in the liquid stream. When the fermentation broth leaves the bioreactor (e.g., under 1-5 bar pressure) to the separator (atmospheric pressure) dissolved gas in the liquid comes out of the liquid and is vented. At least a portion of the gas stream may be removed from the process as tail gas. Tail gases are the gases and vapors which may be released into the atmosphere from a process after all reactions and treatments have taken place.

The separated liquid stream comprises product(s), co-products, and microbial biomass in the aqueous solution of nutrient media. The separated liquid stream is still considered to be fermentation broth, simply with excess gas removed. In order for the fermentation process to operate continuously, the fermentation broth containing microbial biomass in the separated liquid stream from the separator is recycled to the bioreactor for continued use of the microorganisms as biocatalyst. Additional nutrients may be added to the fermentation broth to replenish the medium before it is returned to the gas fermentation bioreactor.

However, before the fermentation broth is recycled to the bioreactor, products are removed and recovered, and co-products are at least partially removed. In general, products may be separated from the fermentation broth using any method or combination of methods known in the art, including, for example, fractional distillation, evaporation, pervaporation, gas stripping, phase separation, extractive separation, including, for example, liquid-liquid extraction. For example, alcohols and/or ketones such as acetone may be recovered by distillation. Acids may be recovered, for example, by adsorption on activated charcoal.

At least a portion of the fermentation broth may be passed serially or in parallel to a vacuum distillation unit and a filtration unit such as a membrane separation unit. The embodiment where the fermentation broth is passed serially from a vacuum distillation unit to a membrane separation is discussed first.

In one embodiment, at least a portion of the fermentation broth, which is the separated liquid stream from the separator is passed to a vacuum distillation unit. The purpose of the separator is to remove the dissolved gas in the fermentation broth so as to “de-gas” the separated liquid stream that enters the vacuum distillation unit. The vacuum distillation unit operates to perform distillation under vacuum, wherein the liquid stream being distilled is enclosed at a low pressure to reduce its boiling point. Suitable vacuum distillation processes are described in U.S. Pat. No. 10,610,802. The vacuum distillation operation separates at least a portion of the liquid stream into a product enriched stream and a product depleted stream. In an embodiment, the product enriched stream comprises ethanol, butyrate, 2,3-butanediol, lactate, butene, butadiene, methyl ethyl ketone, ethylene, acetone, isopropanol, lipids, 3-hydroypropionate, terpenes, terpenoids, isoprene, fatty acids, 2-butanol, 1,2-propanediol, 1-propanol, C6-C12 alcohols, or any mixture thereof. The product depleted stream is enriched in microbial biomass. As used herein, the term “enriched” means that the outlet stream has a greater concentration of the indicated component than in the inlet stream to a vessel. As used herein, the term “depleted” means that the inlet stream has a greater concentration of the indicated component than in the outlet stream of a vessel.

Vacuum distillation is an effective process to recover at least one product from the liquid stream of the fermentation broth while maintaining the viability of the microbial biomass contained in the fermentation broth. “Viability” or “viability of the microbial biomass” refers to the ratio of microorganisms that are alive, capable of living, developing, or reproducing to those that are not. For example, viable microbial biomass in an effluent from a vacuum distillation unit may refer to the ratio of live/dead microorganisms within the vacuum distillation unit effluent. It is advantageous to maintain the highest ratio of viable microorganisms in order to provide the greatest amount of active biocatalyst to the bioreactor. In at least one embodiment, the viability of the microbial biomass in an effluent of the vacuum distillation unit is at least about 80%.

The vacuum distillation unit processes the liquid stream through the use of pressure reduction, where the pressure inside the vacuum distillation unit is maintained below atmospheric so as to lower the temperature needed to vaporize the liquid in the fermentation broth. The pressure of the vacuum distillation unit may be maintained between 40 mbar(a) and 100 mbar(a) to preserve the viability of the microorganisms. In embodiments, the vacuum distillation unit may be maintained at a pressure of from about 40 mbar(a) to about 80 mbar(a), from about 40 mbar(a) to about 90 mbar(a), or from about 45 mbar(a) to about 90 mbar(a). Operating at these pressures results in a temperature gradient within the vacuum distillation vessel where the temperature increases over the length of the vessel, being lowest at the top of the vacuum distillation vessel and highest at the bottom of the vacuum distillation vessel. As the liquid stream flows down the vacuum distillation vessel the concentration of the product is reduced, and therefore the concentration of the product is highest at the top of the vacuum distillation vessel and lowest at the bottom of the vacuum distillation vessel. In an embodiment, the vacuum distillation unit is operated at a temperature in the range from about 35° C. to about 50° C. In certain instances, the temperature may be in the range of from about 40° C. to about 45° C., or from about 37° C. to about 45° C., or from about 45° C. to about 50° C. Additionally, by keeping the residence time, defined as the time that the liquid stream is within the vacuum distillation unit, within a certain period of time, the viability of the microorganisms may be preserved. A residence time of about 0.5 minutes to about 15 minutes allows for the broth to be effectively processed while also preserving the viability of the microorganisms.

In an embodiment, the vacuum distillation unit operates at a lower temperature and pressure, and steam is passed in a counterflow to effectively recover desired volatile organic products from the fermentation broth. The liquid stream of fermentation broth from the separator is passed into the vacuum distillation unit under vacuum and heated with steam, to vaporize the liquid. The product enriched stream is separated from the remaining liquid at a temperature that is low enough to preserve the viability of the microbial biomass, so the microbial biomass may be returned to the bioreactor to continue the production of fermentation products. The product enriched stream is removed from the vacuum distillation unit thereby recovering the desired product. The product enriched stream may be utilized as recovered or may be further processed to achieve the desired purity of the product. The liquid broth, or product depleted stream, generated from the vacuum distillation unit may comprise less than 0.2 wt. % product. In certain embodiments, the product depleted stream comprises less than 1.0 wt. % product. In another embodiment, the product depleted stream comprises between 0.1 and 1.0 wt. % product.

The vacuum distillation temperature for recovering desired products may not be sufficient to also separate other co-products or other metabolites. Since less volatile co-products may not be separated and removed by the vacuum distillation, the less volatile co-products may remain in the liquid broth, i.e., the product depleted stream, along with the microbial biomass, media, and water resulting from steam condensation. Without the removal of co-products, upon recycling of the broth to the bioreactor, the concentration of co-products would continue to increase. The accumulation of co-products in the continuously recycled broth may reach a concentration level that would harm the biocatalyst and negatively affect the fermentation.

In one specific example where ethanol is the desired product and a co-product is 2,3 butanediol (2,3-BDO), the vacuum distillation provides for a separated stream of the target ethanol product, a product enriched stream. However, the co-product, 2,3-BDO, is not separated and remains in the liquid broth removed from the vacuum distillation system as the product depleted stream along with the microbial biomass, media, and water. In a continuous process, where the broth from the vacuum distillation system is continuously recycled to the bioreactor, less volatile co-product 2,3-BDO would accumulate in the broth to a level that potentially negatively affects the fermentation.

One technique to remove co-products from the broth, or product depleted stream, from the vacuum distillation system is to employ a bleed stream. A bleed stream operates to remove and prevent a small portion of the broth from being recycled to the bioreactor. However, a drawback of a bleed stream is that all components of the broth are present in the bleed stream, not just the components desired to be removed. For example, microbial biomass and nutrients in the bleed stream are removed from being recycled to the bioreactor in addition to co-products. Thus, the amount of biocatalyst and nutrients in the bioreactor would be less and may need to be replenished.

Instead of a bleed stream, the disclosure calls for the vacuum distillation unit to be coupled with a membrane separator module allowing for the removal of co-products and water remaining in the broth, the product depleted stream from the vacuum distillation system, whilst retaining the microbial biomass in the broth for recycle to the bioreactor. The use of both the vacuum distillation unit and the membrane separator provides a continuous loop system in which most of the microbial biomass, i.e., microorganisms, are recycled to the bioreactor resulting in minimal loss of the microorganisms due to separation techniques. To maintain the high productivity of the fermentation products, the maximum amount of microbial biomass is desired to be recycled to the bioreactor. The membrane separator may be implemented using crossflow filtration to withdraw permeate containing the undesired co-products whilst recycling the biomass in the broth.

At least a portion of the product depleted stream removed from the vacuum distillation unit, or the fermentation broth from the bioreactor, is passed to the membrane separator. The membrane separator provides a retentate and a permeate. The retentate contains microbial biomass which cannot pass through the membrane. The permeate is depleted in microbial biomass as the microbial biomass cannot pass through the membrane. However, the permeate does contain a co-product which can pass through the membrane. Only the retentate is recycled to the bioreactor. The permeate is removed from the system, and therefore at least a portion of the co-products are removed from the system and not recycled to the bioreactor. Continuously removing co-product from the broth operates to prevent accumulation of the co-product to levels harmful to the fermentation. The membrane separation step may be carried out in one or multiple steps.

As discussed, the membrane separator is a module comprising a membrane filter to separate the product depleted stream into a permeate stream and a retentate stream. Suitable types of membrane separator may include, but are not limited to, hollow fiber membrane, spiral wound membrane, ceramic membrane, and electrodialysis reversal (EDR) membrane. The term “permeate” is used to refer to components of the fermentation broth that pass through the membrane. The permeate stream typically comprises water, remaining soluble fermentation products, co-products, and nutrients. The “retentate” is used to refer to a stream containing components of the fermentation broth that cannot pass through the membrane. The retentate stream comprises the microbial biomass along with water, remaining soluble fermentation products, co-products, and nutrients. At least a portion of the retentate stream is recycled to the bioreactor whereas the permeate stream is removed from the system. As the permeate stream comprises co-products, removal of the permeate stream operates to remove co-products and prevent accumulation of the co-products to a harmful level.

The membrane of the membrane separator may be ceramic or may be made of poly (vinylidene fluoride) (PVDF), polyethylene (PE), PP, poly (vinyl chloride) (PVC), or other polymeric materials. The pore size of the membrane is selected so that the microorganisms cannot pass through the pores of the membrane. The typical pore size of the membrane is in the range of about 0.2 μm to about 10 μm. In one embodiment, the pore size of the membrane may be in the range of about 0.2 μm to about 2 μm. In certain embodiments, the pore size of the membrane may be in the range of about 2 μm to about 5 μm. In another embodiment, the pore size of the membrane may be in the range of about 5 μm to about 10 μm. Suitable ceramic membranes are microporous membranes, used in ultrafiltration and microfiltration applications for which solvent resistance and thermal stability are required. Ceramic membranes are a type of artificial membrane made from inorganic materials, such as alumina, titania, zirconia oxides, silicon carbide, or some glassy materials.

In another embodiment, the fermentation broth is passed to a vacuum distillation unit and a membrane separation unit in parallel. Both the membrane separator and the vacuum distillation unit are separately connected to the bioreactor to provide advantages comprising decreased time for which the microorganisms are in a product depleted stream or in the retentate stream, and high possible broth flux rate through the membrane separation. However, when the membrane separator loop is a separate loop from the vacuum distillation loop and the permeate stream from the membrane separator contains low concentration (e.g., at least 2 wt. %) of a primary product (e.g., ethanol), the process may require a rectifier integrated to downstream of the vacuum distillation unit to recover the primary product.

In another embodiment, both a vacuum distillation unit and a membrane separator are used in series to effectively process the fermentation broth and maintain an advantageous dilution rate of the overall system. As used herein, the term “dilution rate” means the total volume of the liquid flow through a defined portion of the system in a set time period. For example, the dilution rate for a reactor may be the total liquid flow rate per unit volume of the reactor. Vacuum distillation used in conjunction with membrane separation operates to control product and co-product accumulation in the bioreactor at a given feed rate and facilitate the recycle of the microorganisms to the bioreactor. The use of the vacuum distillation unit along with the membrane separator increases the overall productivity and effectiveness of the gas fermentation process. When the membrane separator and the vacuum distillation are in the same loop, the permeate stream from the membrane separator comprising a low concentration of primary product may be passed to the wastewater treatment without needing further product separation to recover the primary product.

As discussed, at least a portion of the product depleted stream, the retentate after membrane separation, is recycled to the bioreactor since the retentate contains microbial biomass. Due to various processing steps, the product depleted stream may have a higher temperature than the operating temperature of the membrane separator or the bioreactor. Therefore, prior to being passed back to the bioreactor, the product depleted stream or the retentate may undergo cooling or heat exchange with a stream needing to be heated. The cooling of the product depleted stream or retentate may be completed by known techniques that reduce the temperature of the stream to achieve a desired temperature. By reducing the temperature of the product depleted stream prior to passing to the membrane separator, or the retentate prior to passing to the bioreactor, unnecessary heating of the culture in the bioreactor can be avoided. The temperature of the fermentation broth within the bioreactor is maintained within an acceptable range, suitable for the culture, in order to maximize the growth and viability of the microorganisms. Thus, monitoring and controlling the temperature of the product depleted stream or retentate is desired.

In an embodiment, the arrangement of the vacuum distillation unit along with the membrane separator forms a continuous loop system. In one embodiment, while operating this loop system, at least a portion of the product depleted stream may be removed as a bleed stream before passing to the membrane separator. The bleed stream comprises microbial biomass. In some embodiments, removing some microbial biomass from the system is advantageous and keeps the amount of microbial biomass in an acceptable range for operation. In another embodiment, the bleed stream may be dried to recover dried microbial biomass which may be used as a protein source, valuable for example, for animal feed. In still another embodiment, the bleed stream may be used to generate energy by burning in burners that heat a unit such as a gasifier, or the bleed stream may be introduced directly into a gasifier to produce additional substrate for the gas fermentation process.

In an embodiment, at least a portion of the permeate stream from the membrane separator may be passed to a wastewater treatment unit to obtain a treated water stream. At least a portion of the treated water stream may be recycled to the bioreactor as a make-up water stream in order to maintain proper levels of broth and operate the fermentation process continuously.

In another embodiment, substantially all of the microbial biomass produced by the fermentation process is recycled to the fermentation process after product recovery, treated by a wastewater treatment unit, and/or sent to a gasification process to produce substrate and/or C1-carbon source. In certain instances, the gasification process receives at least 20%, at least 30%, at least 40%, at least 50%, at least 60%, at least 70%, at least 80%, at least 90%, or substantially all of the microbial biomass from the fermentation process.

Another surprising advantage of the disclosure arises with particular feedstocks and the stoichiometry of gas fermentation. Gas fermentation is very flexible and a wide variety of sources of carbon may be used to produce the gas feedstock. Furthermore, a variety of different gas mixtures may be used as the gas feedstock to the gas fermentation process. Examples of different mixtures, H₂:CO molar ratios, and the associated stoichiometry for gas fermentation to produce ethanol as the primary product are shown in Table 1.

TABLE 1

| | | H₂:CO |
|---|---|---|
| Gases | Stoichiometry | Ratio |
| CO | 6 CO + 3 H₂O → C₂H₅OH + 4 CO₂ | 0:1 |
| CO + H₂ | 3 H₂ + 3 CO → C₂H₅OH + CO₂ | 1:1 |
| CO + H₂ | 4 H₂ + 2 CO → C₂H₅OH + H₂O | 2:1 |
| CO + H₂ + CO₂ | 5 H₂ + 1 CO + 1 CO₂ → C₂H₅OH + 2 H₂O | 5:1 |
| H₂ + CO₂ | 6 H₂ + 2 CO₂ → C₂H₅OH + 3 H₂O | 1:0 |

With some gas mixtures, such as CO+H₂ at 2:1 H₂:CO molar ratio, CO+H₂+CO₂ at 5:1 H₂:CO molar ratio, and H₂+CO2 at 1:0 H₂:CO molar ratio, the stoichiometry shows that water is produced. Under conditions where water is produced in the gas fermentation, the disclosure provides a surprising benefit in controlling the liquid level in the continuous process. The bioreactor is typically a fixed volume vessel. In continuous operation with recycle, without a control mechanism, if produced in the gas fermentation, water would accumulate in the system. Such accumulation would approach and potentially exceed the volume of the system and the capacity of the bioreactor in particular. If a system were to employ vacuum distillation without membrane separation, the product, such as ethanol, would be removed from the circulating broth, but water would continue to accumulate increasing the volume of circulating broth. Furthermore, vacuum distillation itself adds water to the system by using steam to heat the broth for distillation. The more ethanol produced in gas fermentation; the more steam is required for the vacuum distillation separation. Some of the steam condenses and adds to the volume of water in the liquid broth. As discussed above, a bleed stream is an option to control the liquid level in the circulating broth. However, a bleed stream removes valuable microbial biomass which then may need to be replaced. The disclosure calls for a membrane separator which has the added advantage of providing control of the liquid level in the system so as not to overwhelm the set volume of the equipment. The membrane separator provides a permeate stream comprising the liquid portion of the broth including water generated by the gas fermentation but not including the microbial biomass. The permeate stream may be removed from the system thereby controlling the amount of liquid in the broth that is circulated through the continuous process. Yet the microbial biomass is preserved in the broth which is particularly advantageous in embodiments where the microorganisms are slower to grow and therefore harder to replenish. In some embodiments, a combination of a bleed stream and the membrane separator may be the best option. The bleed stream may be adjusted as appropriate to maintain liquid levels and biocatalyst levels in the system.

In some embodiments, the presence of H₂ may be beneficial to the gas fermentation process. When employing hydrogen as a component of the substrate, it is advantageous to use hydrogen that has been produced with no harmful greenhouse gas emissions. This type of hydrogen can be made using clean electricity from renewable energy sources such as solar or wind in order to electrolyze water. Other non-fossil sources of energy have also been used to generate hydrogen through electrolysis. Electrolysis of water uses an electrochemical reaction to split water into its components of hydrogen and oxygen without emitting CO₂ in the process. The reaction involved in water electrolysis is 2 H₂O+electricity→2 H₂+O₂+heat. Water electrolysis technologies are known, and exemplary processes include alkaline water electrolysis, protein exchange membrane (PEM) electrolysis, and solid oxide electrolysis. In another embodiment, hydrogen may be produced from natural gas using a process typically referred to as steam reforming. Steam reforming is defined as the general process by which hydrogen is produced and recovered by the catalytic reaction of a hydrocarbon feedstock, typically methane, and steam according to the reaction CH₄+H₂O→CO+3H₂. Carbon monoxide is also produced and may be utilized in the gas fermentation process and thus not released into the atmosphere. Carbon dioxide produced in the steam reforming process through subsequent water-gas shift reaction may also be utilized in the gas fermentation process and not released into the atmosphere.

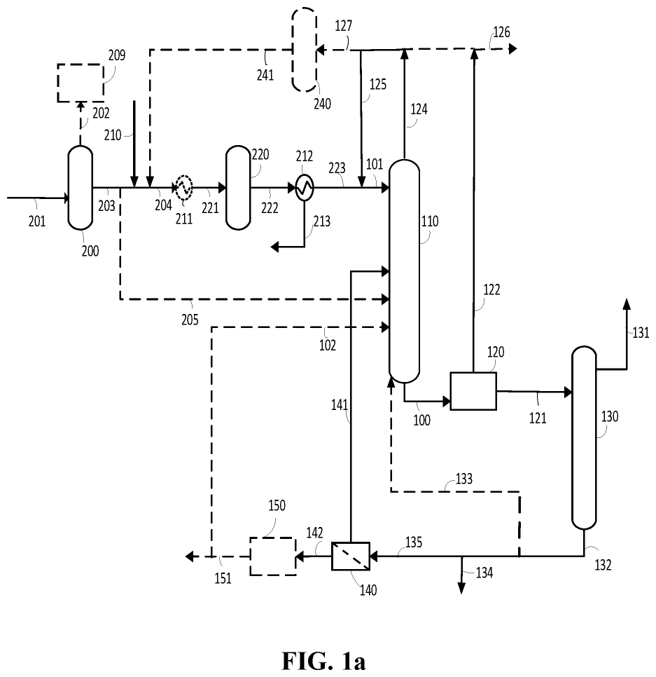

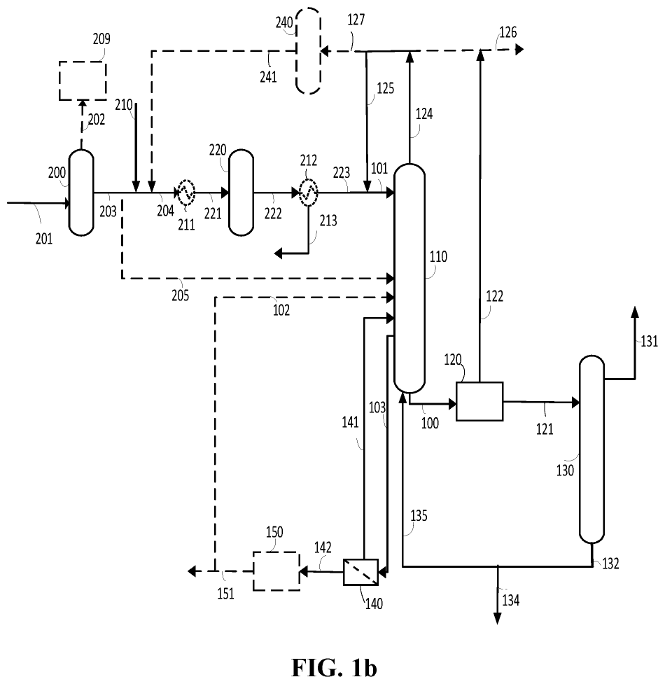

For a better understanding of the present disclosure, reference is made to the following examples and figures. Some embodiments may be described by reference to the process configurations shown in FIG. 1a and FIG. 1b, which relates to both apparatus and process. Any reference to a process step includes reference to an apparatus unit or equipment that is suitable to carry out the step, and vice-versa. The following detailed description is merely exemplary and is not intended to limit the application and uses of the embodiment described.

In FIG. 1a and FIG. 1b, water stream 201 is passed to hydrogen generation unit 200. Hydrogen generation unit 200 may be an electrolyzer, a steam reformer, or a combination thereof. In an embodiment, hydrogen generation unit 200 generates O₂ containing stream 202 and hydrogen stream 203 comprising at least H₂. O₂ containing stream 202 may be passed to thermal oxidizer 209 to generate steam. The steam generated may be used to produce electricity or may be used as a source of energy elsewhere in the process or in another process. The O₂ produced as a by-product of hydrogen generation unit 200 provides additional benefits beyond gas fermentation.

C1 containing gas stream 210 comprises CO₂ and/or CO which may be sourced from domestic, industrial, or agricultural waste or by-product. C1 containing gas stream 210 may be combined with at least a portion of hydrogen stream 203 to obtain gas fermentation feedstock 204.

Gas fermentation feedstock 204 may optionally be passed through heating unit 211 to generate heated gas fermentation feedstock 221 which is passed to an optional CO₂ to CO conversion system 220. The CO₂ to CO conversion system 220 operates to increase the amount of CO and decrease the amount of CO₂ in gas fermentation feedstock 221 to provide adjusted gas fermentation feedstock 222 comprising CO, CO₂. and H₂. Adjusted gas fermentation feedstock 222 has a higher relative concentration of CO as compared to gas fermentation feedstock 204 or heated gas fermentation feedstock 221. CO₂ to CO conversion system 220 is at least one system selected from reverse water gas shift reaction system, CO₂ electrolysis system, thermo-catalytic conversion system, electro-catalytic conversion system, partial combustion system, plasma conversion system, or any combination thereof. In one embodiment, the CO₂ to CO conversion system 220 may be a reverse water gas shift reaction system that produces water from carbon dioxide and hydrogen, with carbon monoxide as a side product. The composition of CO and H₂ is defined as water gas. The term ‘shift’ in water-gas shift means changing the water gas composition (CO and H₂ ratio). The ratio can be increased by adding CO₂ or reduced by adding steam to the system. The reverse water gas shift reaction system may comprise a single stage or more than one stage, and different stages may be conducted at different temperatures and may use different catalysts. In another embodiment, the CO₂ to CO conversion system 220 uses thermo-catalytic conversion to disrupt the stable atomic and molecular bonds of CO₂ and other reactants over a catalyst using thermal energy as the driving force of the reaction to produce CO. Since CO₂ molecules are thermodynamically and chemically stable, if CO₂ is used as a single reactant, large amounts of energy are required. Therefore, often other substances such as hydrogen are used as a co-reactant to make the thermodynamic process easier. Many catalysts are known for the thermo-catalytic process such as metals and metal oxides as well as nano-sized catalyst metal-organic frameworks. Various carbon materials have been employed as carriers for thermocatalytic catalysts. In another embodiment, the CO₂ to CO conversion system 220 uses electro-catalytic conversion for the electrocatalytic reduction of carbon dioxide to produce synthesis gas from water and carbon dioxide. Such electro-catalytic conversion, also referred to as electrochemical conversion, of carbon dioxide typically involves electrodes in an electrochemical cell having a solution supporting an electrolyte through which carbon dioxide is bubbled. The synthesis gas, also known as syngas, produced comprises CO, and is separated from the solution of the electrochemical cell and removed. In another embodiment, the CO₂ to CO conversion system 220 uses the combination of photocatalysis and electrocatalysis in photoelectrocatalysis which uses for example sunlight irradiation.

Some CO₂ to CO conversion systems 220 operate at elevated temperatures, and therefore combined gas stream 204 may be passed through optional heating unit 211 to obtain heated combined stream 221. Heated combined stream 221 may be at a temperature in the range of about 400° C. to about 600° C. before being passed to CO₂ to CO conversion system 220. In CO₂ to CO conversion system 220, at least a portion of CO₂ present in heated combined stream 221 is converted to CO to generate gaseous feed stream 222 enriched in CO, comprising a concentration of CO that is greater than that of heated combined stream 221. As gas fermentation in bioreactor 110 may be conducted at a temperature less than needed in CO₂ to CO conversion system 220, gaseous feed stream 222 from CO₂ to CO conversion system 220 may be passed to optional cooling unit 212 to obtain cooled gaseous feed stream 223. Water may condense in the cooling process and by-product water stream 213 may be generated. Cooled gaseous feed stream 223 may be at a temperature in the range of about 35° C. to about 55° C. Cooled gaseous feed stream 223 may be passed directly to bioreactor 110. Cooled gaseous feed stream 223 may be combined with gas recycle stream 125 to form bioreactor feed 101 which is introduced to bioreactor 110. In an embodiment, at least a portion 205 of the first gas stream 203 comprising H₂ may be passed to bioreactor 110. In this way, the total volume of material passed through CO₂ to CO conversion system 220 may be minimized as it may not be required to pass all of the first gas stream 203 through CO₂ to CO conversion system 220 in order to achieve the desired amount of CO thus saving cost and reducing the size of the conversion system 220. Further, the amount of hydrogen passed to bioreactor 110 may be more precisely controlled through the use of stream 205. Similarly, a portion of combined gas stream 204 may be passed directly to bioreactor 110, bypassing (not shown) CO₂ to CO conversion system 220.

Cooled gaseous feed stream 223, or bioreactor feed 101, after optional mixing with gas recycle stream 125, is introduced to bioreactor 110 as a substrate for the microorganism biocatalyst which is present in a nutrient solution. Gas fermentation proceeds and the product is produced. Fermentation broth stream 100 comprising at least microbial biomass of the biocatalyst, nutrient solution, and the product is passed to separator 120. Separator 120 operates to separate a gas stream 122 from the fermentation broth stream 120 and to obtain a liquid-based stream 121. The liquid-based stream 121 comprises the nutrient solution and the microbial biomass. Gas stream 124 from the bioreactor 110 is separated. At least a portion 125 of the gas stream 124 is recycled to the bioreactor feed 101. In an embodiment, at least a portion 126 of gas stream 122 may be removed from the process as tail gas in order to, for example, prevent the buildup of inert gases. In another embodiment, at least a portion 127 of the gas stream 124 may be passed to gas treatment unit 240 to remove contaminants such as benzene, toluene, xylene, ethyl benzene, or other fermentation inhibitors or by-products of gas fermentation. Gas treatment unit 240 may be an activated carbon bed unit, an adsorption unit, or any combination thereof. Treated gas stream 241 is produced from gas treatment unit 240 and is depleted in undesired components. Treated gas stream 241 may be recycled and combined with first gas stream 203 (not shown), second gaseous stream 210 (not shown), heated combined stream 221 (not shown), gaseous feed stream 222 (not shown), cooled gaseous feed stream 223 (not shown), or combined gas stream 204 as shown. It is also envisioned that treated gas stream 241 may replace gas recycle stream 125.

The liquid stream 121 is passed to vacuum distillation unit 130 and separated by vacuum distillation into product enriched stream 131 and product depleted stream 132. Vacuum distillation unit 130 effectively recovers product from the fermentation broth and prevents product accumulation in the bioreactor. At the same time, the vacuum distillation unit provides the separation at a temperature suitable for maintaining the viability of the microbial biomass. In one embodiment, the viability of the microbial biomass in the product depleted stream 132 is greater than 85 percent. In another embodiment, the viability of the microbial biomass in product depleted stream 132 is within 5 to 10 percent of that found in liquid stream 121 entering vacuum distillation unit 130. Product depleted stream 132 has reduced proportions of product compared to liquid stream 121 such that product accumulation within the bioreactor is effectively reduced. In an embodiment, product depleted stream 132 comprises less than 0.2 wt. % product. An example of a suitable vacuum distillation unit and operation is found in U.S. Pat. Nos. 10,610,802 and 11,471,786.

Vacuum distillation unit 130 is designed to preserve the viability of the microbial biomass. Therefore, the product depleted stream being passed to the bioreactor contains viable biocatalyst to continue the gas fermentation process. The microbial biomass viability is maintained at a sufficiently high percentage. At least a portion 134 of the product depleted stream 132 may be removed as bleed stream to, for example, control the amount of biocatalyst in the bioreactor.

In an embodiment, the desired product in the product enriched stream 131 is ethanol. To be able to achieve high ethanol productivity with gas fermentation, the maximum amount of microbial biomass may be required in the bioreactor. To achieve a high ethanol production rate, vacuum distillation unit 130 also provides continuous ethanol product removal. In vacuum distillation unit 130, liquid stream 121 is placed under vacuum and heated with steam, in order to vaporize and strip ethanol at a temperature that is low enough to preserve the viability of microbial biomass. The microbial biomass may then be returned to the bioreactor 110 to continue ethanol production. However, the recovery of ethanol by vacuum distillation may result in the accumulation of less volatile co-products, such as 2,3 butanediol (2,3 BDO), nutrients, and metabolites in the liquid product-depleted stream 132 removed from the bottom of vacuum distillation unit 130. Without further processing steps, the less volatile co-products may be recycled to bioreactor 110 and with each pass through the overall cycle, may continuously accumulate in the liquid phase. The same accumulation may occur with water in the system. Water in the system may increase due to the fermentation reaction in bioreactor 110 and from steam condensate in vacuum distillation unit 130. Therefore, the liquid level in the bioreactor 110 may rise. As bioreactor 110 is a fixed volume, it is advantageous to maintain liquid levels in bioreactor 110. Example 1 below demonstrates the accumulation of 2,3 BDO co-product and the accumulation of liquid in the bioreactor when vacuum distillation is used.

### Example 1: Vacuum Distillation was Employed to Recover Ethanol

Gaseous feed stream comprising a predetermined amount of CO₂ and CO was passed to the bioreactor to generate a fermentation broth. The fermentation broth consisted of microbial biomass, ethanol, acetate, 2,3 BDO, and water. The bioreactor was operated under conditions for the fermentation of a C1-containing gas. The fermentation broth was passed to separator and got separated into a gas stream and a liquid stream. The liquid stream separated from the separator was passed to vacuum distillation unit. The concentration of microbial biomass in the liquid stream was measured by a spectrophotometer method (OD600), and vacuum distillation was employed to recover ethanol.

Over the course of around a two-weeks gas fermentation run, the concentration level of biomass was constant at about 5 g/L for a steady state of growth and decay of the biomass. The ethanol concentration was level at about 18 g/L. The concentration level of acetate was maintained at about 4 g/L due to the metabolic regulation of the biocatalyst. The bleed stream was adjusted to maintain predetermined liquid levels and microbial biomass levels in the system. Noticeably, the co-product of 2,3 BDO continued to increase in concentration and reached 17 g/L. This increasing concentration of 2,3 BDO was because 2,3 BDO is a less volatile co-product as compared to ethanol, therefore, 2,3 BDO was not stripped via vacuum distillation and was not metabolically regulated. With no liquid removal other than from bleed withdrawal at a low dilution rate of around 0.2/day, there was no way to remove the co-products 2,3 BDO and water. This accumulation effect may also apply nutrients fed at a rate greater than their specific consumption rate.

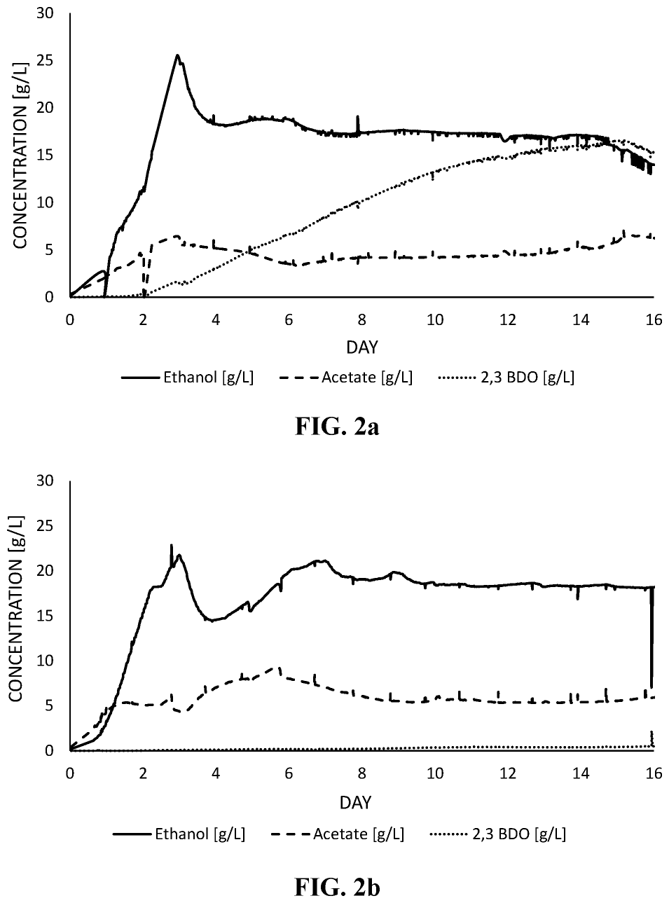

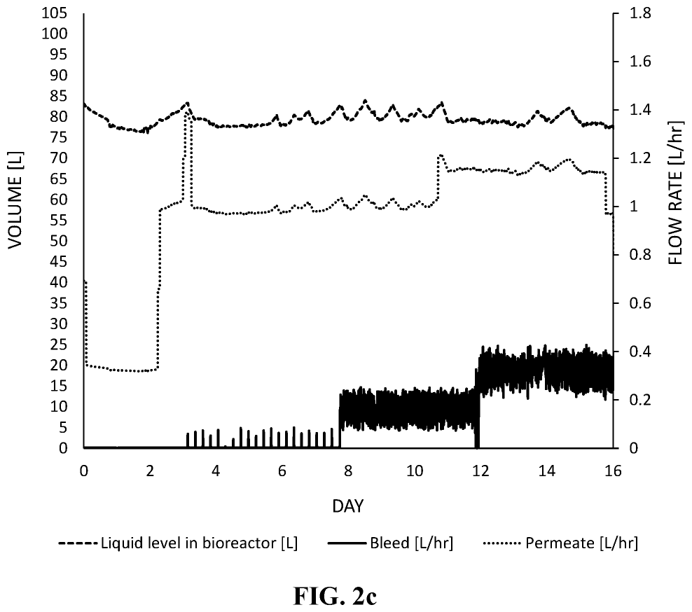

FIG. 2a is depicting a plot showing the concentration of ethanol, acetate, and 2,3 BDO when vacuum distillation was employed for around two weeks in the gas fermentation run of Example 1. The plot is depicting that ethanol concentration (solid line) and the acetate concentration (dash line) were approximately constant at a level but the co-product 2,3 BDO (dotted line) continued to increase in concentration over the course of the run.

To overcome the accumulation of less volatile co-products and to control the liquid level in bioreactor 110, vacuum distillation unit 130 is coupled with membrane separator 140 as shown in FIG. 1a and FIG. 1b. These arrangements provide for the removal of less volatile co-products, such as 2,3 BDO, and the controlling of liquid level in the system whilst retaining the recycling of microbial biomass to the bioreactor 110. In FIG. 1a, at least a portion 133 of the product depleted stream 132 may be recycled to bioreactor 110. The portion 133 of the product depleted stream 132 may have a higher than acceptable temperature as compared to the temperature of the bioreactor, and thus the portion 133 recycled to bioreactor 110 may require cooling (not shown) prior to being passed to bioreactor 110. At least a portion 135 of the product depleted stream 132 is passed to membrane separator 140 to separate the stream into retentate stream 141 and permeate stream 142. The retentate stream 141 comprises microbial biomass that cannot permeate the membrane and water. The permeate stream typically comprises water and soluble fermentation products, co-products, and nutrients. The retentate stream 141 is recycled to bioreactor 110 to enrich the microbial biomass inside the bioreactor 110. In an embodiment, permeate stream 142 may be passed to wastewater treatment unit 150 to remove non-water components from the permeate stream thereby providing treated water stream 151. At least a portion of treated water stream 151 may be passed to the bioreactor 110 as make-up water stream 102. In FIG. 1b, the product depleted stream 135 is recycled to the bioreactor 110. At least a portion 103 of the fermentation broth is passed to membrane separator 140 to separate the stream into retentate stream 141 and permeate stream 142. The retentate stream 141 comprises microbial biomass that cannot permeate the membrane and water. The permeate stream typically comprises water and soluble fermentation products, co-products, and nutrients. The retentate stream 141 is recycled to bioreactor 110 to enrich the microbial biomass inside the bioreactor 110. In an embodiment, permeate stream 142 may be passed to wastewater treatment unit 150 to remove non-water components from the permeate stream thereby providing treated water stream 151. At least a portion of treated water stream 151 may be passed to the bioreactor 110 as make-up water stream 102. Example 2 below demonstrates a fermentation run by the use of vacuum distillation 130 and membrane separator 140.

### Example 2: Vacuum Distillation and Membrane Separator were Employed to Recover Ethanol

Gaseous feed stream comprising a predetermined amount of CO₂ and CO was passed to bioreactor to generate a fermentation broth. The fermentation broth was comprised of microbial biomass, ethanol, acetate, 2,3 BDO, and water. The bioreactor was operated under conditions for the fermentation of a C1-containing gas. The fermentation broth was passed to separator and separated into a gas stream and a liquid stream. The liquid stream separated from the separator was passed to vacuum distillation unit and separated into ethanol enriched stream and ethanol depleted stream enriched in microbial biomass. A bleed stream was removed from the ethanol-depleted stream after vacuum distillation. The ethanol depleted stream was passed to membrane separator and separated into permeate stream and a retentate stream.

In this gas fermentation run, both vacuum distillation and membrane separator were employed to recover ethanol. To be able to achieve more ethanol productivity with gas fermentation, the maximum amount of microbial biomass was required in the bioreactor. So, in this gas fermentation run, the membrane separator was implemented to withdraw permeate comprising 2, 3 BDO, and water whilst recycling the microbial biomass as a retentate to the bioreactor. The membrane separator employed with the vacuum distillation unit provided continuous ethanol recovery.

Over the course of around a two-weeks gas fermentation run, the ethanol concentration level was at about 20 g/L and the concentration level of acetate was at about 5 g/L, but 2,3 BDO was not accumulated. Bleed stream was removed from the ethanol depleted stream before passing it to the membrane separator. The bleed withdrawal contributed to a dilution rate of 0.12/day. The permeate withdrawal contributed to an additional 0.27/day dilution rate, for a combined total of 0.39/day overall dilution rate for the analyzed period of day 10 to day 20. The total dilution rate when both vacuum distillation and membrane separator were employed was approximately double the dilution rate when only vacuum distillation was employed. The addition of the membrane separator to the vacuum distillation unit allowed for increasing the total dilution rate by enabling a permeate flow rate. The total dilutional rate of the system is the sum dilution rate of the permeate stream and the dilution rate of the bleed stream. The use of vacuum distillation and membrane separator together allowed for independent control of the bleed stream.

FIG. 2b is depicting a plot showing the concentration of ethanol, acetate, and 2,3 BDO when both vacuum distillation and membrane separator were employed for around two weeks in the gas fermentation run of example 2. The plot of FIG. 2b is depicting that ethanol concentration (solid line) and the acetate concentration (dash line) were approximately constant at a level and the co-product 2,3 BDO (dotted line) was not accumulated over the course of the run.

In accordance with the present disclosure, the stoichiometry for ethanol shows that with some gas mixtures, such as CO+H₂, CO+H₂+CO₂. and H₂+CO₂ at an H₂:CO molar ratio greater than 1:0, water is produced in the gas fermentation. In continuous operation with a low dilution rate, achievable by employing vacuum distillation to achieve a high liquid recycle rate, without a control mechanism, if produced in the gas fermentation, water would accumulate in the system. Vacuum distillation itself adds water to the system by using steam to heat the fermentation broth for distillation. Bleed stream, an option to control the liquid level in the circulating broth, removes valuable microbial biomass which then may need to be replaced. In Example 2, when both the vacuum distillation unit and the membrane separator were employed, the added advantage of control of the liquid level in the system was also monitored.

FIG. 2c is depicting a plot showing control of the liquid level in the system when a gaseous feed stream having an H₂:CO molar ratio of 5:1 was fed to a continuous gas fermentation run of Example 2. The plot of FIG. 2c is depicting that the liquid level in the bioreactor (dash line) was stable despite water production in the bioreactor from the reaction as well as from the steam condensation in the vacuum distillation unit. When the membrane separator was employed with the vacuum distillation unit, permeate (dotted line) was drawn off and allowed independent control of the bleed rate (solid line). It shows that a bleed flow rate alone would not have been sufficient to maintain a stable liquid level. Permeate was required. For example, at day 14 bleed would need to be increased to 1.5 L/h to maintain the liquid level without drawing permeate (Bleed of 0.35/h+permeate of 1.15/h=1.5 L/h), which would cause biomass washout.

The use of the vacuum distillation unit with the membrane separator provides efficient product recovery, cell retention, and water recycling to maximize the production of the fermentation products and bring down wastewater treatment and water footprint to bring down overall CAPEX. The use of the vacuum distillation unit with the membrane separator offers multiple other advantages including i) a reduction in the required dilution rate thus lowering the total water footprint and retaining nutrients resulting in environmental and cost benefits, and ii) independent control of product concentration which keeps the microbial biomass from reaching a stressful or toxic environment at high product concentration.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavor in any country.

The use of the terms “a” and “an” and “the” and similar terms are to be construed to cover both the singular and the plural, unless otherwise indicated herein or clearly contradicted by context. The terms “comprising,” “having,” “including,” and “containing” are to be construed as open-ended terms unless otherwise noted. The use of the alternative, such as the term “or”, should be understood to mean either one, both, or any combination thereof of the alternatives. As used herein, the term “about” means ±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Embodiments of the Disclosure

Embodiment 1. A process for continuous gas fermentation comprising: generating, by gas fermentation of a gas stream in a bioreactor, a fermentation broth comprising gas, microbial biomass, at least one product, and at least one co-product; passing at least a portion of the fermentation broth to a separator and separating a gas stream from a liquid stream comprising the microbial biomass, the at least one co-product, and the at least one product; passing, serially or in parallel, at least a portion of the liquid stream to a vacuum distillation unit and a membrane separation unit; separating, in the vacuum distillation unit, a product enriched stream and a microbial biomass and co-product enriched stream; separating, in the membrane separation unit, a permeate stream enriched in water and co-product and a retentate stream enriched in microbial biomass; and recycling at least a portion of the retentate stream to the bioreactor.

Embodiment 2. The process of embodiment 1 further comprising passing to the bioreactor, at least a portion of the microbial biomass and co-product enriched stream, at least a portion of the retentate, or both.

Embodiment 3. The process of embodiment 1 or embodiment 2 further comprising removing as a bleed stream, at least a portion at least a portion of the microbial biomass and co-product enriched stream, at least a portion of the retentate, or both.

Embodiment 4. The process of any of embodiments 1 to 3 further comprising passing at least a portion of the permeate stream to a wastewater treatment unit, generating a treated water stream, and passing at least a portion of the treated water stream to the bioreactor.

Embodiment 5. The process of any of embodiments 1 to 4 further comprising passing at least a portion of the gas stream to a gas treatment unit and removing at least one contaminant from the gas stream fermenting in the bioreactor.

Embodiment 6. The process of any of embodiments 1 to 5 wherein the microbial biomass and co-product enriched stream comprises less than 0.2 wt. % product.

Embodiment 7. The process of any of embodiments 1 to 6 wherein the vacuum distillation unit is operated at a pressure in the range of about 40 mbar(a) to about 100 mbar(a).

Embodiment 8. The process of any of embodiments 1 to 7 wherein the vacuum distillation unit is operated at a temperature in the range of about 35° C. to about 50° C.

Embodiment 9. The process of any of embodiments 1 to 8, wherein the product is at least one selected from ethanol, acetate, butanol, butyrate, 2,3-butanediol, lactate, butene, butadiene, methyl ethyl ketone (2-butanone), ethylene, acetone, isopropanol, lipids, 3-hydroxypropionate (3-HP), terpenes, terpenoids, isoprene, fatty acids, 2-butanol, 1,2-propanediol, 1 propanol, chorismate-derived products, 3 hydroxybutyrate, and 1,3-butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, monoethylene glycol, 2-phenylethanol, ethylene, or any combination thereof.

Embodiment 10. The process of any of embodiments 1 to 9 wherein the gas stream comprises at least hydrogen and carbon monoxide wherein at least a portion of the carbon monoxide is generated by conversion of carbon dioxide to carbon monoxide.

Embodiment 11. The process of any of embodiments 1 to 10 wherein the conversion of carbon dioxide comprises heating a gas stream comprising carbon-dioxide to a temperature in the range of about 400° C. to about 600° C., contacting with a conversion catalyst, and then cooling the resulting stream comprising carbon monoxide to a temperature in the range of about 35° C. to about 55° C.

Embodiment 12. The process of any of embodiments 1 to 11 wherein the conversion employs reverse water gas shift conversion, CO₂ electrolysis conversion, thermo-catalytic conversion, electro-catalytic conversion, partial combustion conversion, plasma conversion, or any combination thereof.

Embodiment 13. The process of any of embodiments 1 to 12 wherein the carbon dioxide is generated by steam reforming, electrolysis powered by a renewable power source, or both.

Embodiment 14. An apparatus for continuous gas fermentation comprising: a bioreactor comprising a bioreactor inlet, at least one recycle inlet, and a fermentation broth outlet; a separator in fluid communication with the fermentation broth outlet, the separator comprising a liquid outlet and a gas outlet; a vacuum distillation unit and a membrane separator in fluid communication, serially or in parallel, with the separator liquid outlet; the vacuum distillation unit comprising an overhead outlet and a bottoms outlet; and the membrane separator comprising a permeate outlet and a retentate outlet, the retentate outlet in fluid communication with at least one of the bioreactor recycle inlets.

Embodiment 15. The apparatus of embodiment 14 wherein the bottoms outlet is further in fluid communication with at least one of the bioreactor recycle inlets.

Embodiment 16. The apparatus of embodiment 14 or embodiment 15 further comprising a wastewater treatment unit in fluid communication with the permeate outlet and at least one of the bioreactor recycle inlets.

Embodiment 17. The apparatus of any of embodiments 14 to 16 apparatus further comprising a gas treatment unit in fluid communication with the inlet the bioreactor.

Embodiment 18. The apparatus of any of embodiments 14 to 17 further comprising a gas treatment unit in fluid communication with both the gas outlet and at least one of the bioreactor recycle inlets.

Embodiment 19. The apparatus of any of embodiment 14 to 18 further comprising, in fluid communication the bioreactor inlet, a CO₂ to CO conversion unit selected from a reverse water gas reaction unit, a CO₂ electrolysis unit, a thermo-catalytic conversion unit, an electro-catalytic conversion unit, a partial combustion unit, a plasma conversion unit or any combination thereof.

Embodiment 20. The apparatus of any of embodiment 14 to 19 further comprising a bleed conduit in fluid communication with the liquid outlet, retentate outlet, or both.

Embodiment 1a. A process for continuous gas fermentation comprising
a. generating, by gas fermentation in a bioreactor, a fermentation broth comprising gas, microbial biomass, at least one product, and at least one co-product;
b. passing at least a portion of the fermentation broth to a separator and separating into a gas stream and a liquid stream comprising the microbial biomass, the at least one co-product, and the at least one product;
c. passing at least a portion of the liquid stream to a vacuum distillation unit and separating into a product enriched stream and a product depleted stream wherein the product depleted stream is enriched in microbial biomass and at least one co-product;
d. passing at least a portion of the product depleted stream to a membrane separator and separating into a permeate stream enriched in water and at least one co-product and a retentate stream enriched in microbial biomass; and
e. recycling at least a portion of the retentate stream to the bioreactor.

Embodiment 2a. The process of embodiment 1a further comprises passing at least a portion of the product depleted stream to the bioreactor.

Embodiment 3a. The process of any of embodiments 1a or 2a further comprises removing at least a portion of the product depleted stream as a bleed stream.

Embodiment 4a. The process of any of embodiments 1a to 3a further comprises at least a portion of the permeate stream to a wastewater treatment unit to obtain a treated water stream and passing at least a portion of the treated water stream to the bioreactor.

Embodiment 5a. The process of any of embodiments 1a to 4a further comprising at least a portion of the gas stream to a gas treatment unit before passing to the bioreactor.

Embodiment 6a. The process of any of embodiments 1a to 5a further comprising passing at least a portion of fermentation broth to the membrane separator

Embodiment 7a. The process of any of embodiments 1a to 6a wherein the product depleted stream comprises less than 0.2 wt. % product.

Embodiment 8a. The process of any of embodiments 1a to 7a wherein the vacuum distillation unit is operated at a pressure in the range of about 40 mbar(a) to about 100 mbar(a).

Embodiment 9a. The process of any of embodiments 1a to 8a wherein the vacuum distillation unit is operated at a temperature in the range of about 35° C. to about 50° C.

Embodiment 10a. The process of any of embodiments 1a to 9a wherein the product is at least one selected from ethanol, acetate, butanol, butyrate, 2,3-butanediol, lactate, butene, butadiene, methyl ethyl ketone (2-butanone), ethylene, acetone, isopropanol, lipids, 3-hydroxypropionate (3-HP), terpenes, terpenoids, isoprene, fatty acids, 2-butanol, 1,2-propanediol, 1 propanol, chorismate-derived products, 3 hydroxybutyrate, and 1,3-butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, monoethylene glycol, 2-phenylethanol, or any combination thereof.

Embodiment 11a. The process of any of embodiments 1a to 10a further comprising
i. obtaining a first gaseous stream comprising H₂ and passing at least a portion of the first gaseous stream to the bioreactor; and
ii. obtaining a second gaseous stream comprising CO₂ and passing the second gaseous stream to a CO₂ to CO conversion system to obtain a gaseous feed stream enriched in CO and passing the gaseous feed stream to the bioreactor.

Embodiment 12a. The process of any of embodiments 1a to 11a further comprises heating the second gaseous stream to a temperature in the range of about 400 to about 600° C. before passing to the CO₂ to CO conversion system and cooling the gaseous feed stream to a temperature in the range of about 35 to about 55° C. before passing to the bioreactor.

Embodiment 13a. The process of any of embodiments 1a to 12a wherein the CO₂ to CO conversion system is selected from reverse water gas reaction system, CO₂ electrolysis system, thermo-catalytic conversion system, electro-catalytic conversion system, partial combustion system, plasma conversion system, or any combination thereof.

Embodiment 14a. The process of any of embodiments 1a to 13a wherein the first gaseous stream is generated by steam reforming, electrolysis, or both, wherein the electrolysis is powered by a renewable power source.

Embodiment 15a. The process of any of embodiments 1a to 14a further comprising combining at least a portion of the first gaseous with the second gaseous stream.

Embodiment 16a. An apparatus for continuous gas fermentation comprising
a. a bioreactor comprising a bioreactor inlet and a fermentation broth outlet;
b. a separator in fluid communication with the fermentation broth outlet, the separator comprising a liquid outlet;
c. a vacuum distillation unit in fluid communication with the liquid outlet, the vacuum distillation unit comprising an overhead outlet and a bottoms outlet; and
d. a membrane separator in fluid communication with the bottoms outlet, the membrane separator comprising a permeate outlet and a retentate outlet, the retentate outlet in fluid communication with the bioreactor

Embodiment 17a. The apparatus of embodiment 16a wherein the bottoms outlet is further in fluid communication with the bioreactor.

Embodiment 18a. The apparatus of any of embodiments 16a or 17a further comprising a wastewater treatment unit in fluid communication with the permeate outlet and the bioreactor.

Embodiment 19a. The apparatus of any of embodiments 16a to 18a further comprises a gas treatment unit in fluid communication with the gas outlet and the bioreactor.

Embodiment 20a. The apparatus of any of embodiments 16a to 19a further comprises a CO₂ to CO conversion system in fluid communication with bioreactor wherein the CO₂ to CO conversion system is selected from reverse water gas reaction system, CO₂ electrolysis system, thermo-catalytic conversion system, electro-catalytic conversion system, partial combustion system, plasma conversion system, or any combination thereof.

1. A process for continuous gas fermentation comprising:
a. generating, by gas fermentation of a gas stream in a bioreactor, a fermentation broth comprising gas, microbial biomass, at least one product, and at least one co-product;
b. passing at least a portion of the fermentation broth to a separator and separating a gas stream from a liquid stream comprising the microbial biomass, the at least one co-product, and the at least one product;
c. passing, serially or in parallel, at least a portion of the liquid stream to a vacuum distillation unit and a membrane separation unit;
d. separating, in the vacuum distillation unit, a product enriched stream and a microbial biomass and co-product enriched stream;
e. separating, in the membrane separation unit, a permeate stream enriched in water and co-product and a retentate stream enriched in microbial biomass; and
f. recycling at least a portion of the retentate stream to the bioreactor.

2. The process of claim 1 further comprising passing to the bioreactor, at least a portion of the microbial biomass and co-product enriched stream, at least a portion of the retentate, or both.

3. The process of claim 1 further comprising removing as a bleed stream, at least a portion at least a portion of the microbial biomass and co-product enriched stream, at least a portion of the retentate, or both.

4. The process of claim 1 further comprising passing at least a portion of the permeate stream to a wastewater treatment unit, generating a treated water stream, and passing at least a portion of the treated water stream to the bioreactor.

5. The process of claim 1 further comprising passing at least a portion of the gas stream to a gas treatment unit and removing at least one contaminant from the gas stream fermenting in the bioreactor.

6. The process of claim 1 wherein the microbial biomass and co-product enriched stream comprises less than 0.2 wt. % product.

7. The process of claim 1 wherein the vacuum distillation unit is operated at a pressure in the range of about 40 mbar(a) to about 100 mbar(a).

8. The process of claim 1 wherein the vacuum distillation unit is operated at a temperature in the range of about 35° C. to about 50° C.

9. The process of claim 1, wherein the product is at least one selected from ethanol, acetate, butanol, butyrate, 2,3-butanediol, lactate, butene, butadiene, methyl ethyl ketone (2-butanone), ethylene, acetone, isopropanol, lipids, 3-hydroxypropionate (3-HP), terpenes, terpenoids, isoprene, fatty acids, 2-butanol, 1,2-propanediol, 1 propanol, chorismate-derived products, 3 hydroxybutyrate, and 1,3-butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, monoethylene glycol, 2-phenylethanol, ethylene, or any combination thereof.

10. The process of claim 1 wherein the gas stream comprises at least hydrogen and carbon monoxide wherein at least a portion of the carbon monoxide is generated by conversion of carbon dioxide to carbon monoxide.

11. The process of claim 10 wherein the conversion of carbon dioxide comprises heating a gas stream comprising carbon-dioxide to a temperature in the range of about 400° C. to about 600° C., contacting with a conversion catalyst, and then cooling the resulting stream comprising carbon monoxide to a temperature in the range of about 35° C. to about 55° C.

12. The process of claim 10 wherein the conversion employs reverse water gas shift conversion, CO₂ electrolysis conversion, thermo-catalytic conversion, electro-catalytic conversion, partial combustion conversion, plasma conversion, or any combination thereof.

13. The process of claim 10 wherein the carbon dioxide is generated by steam reforming, electrolysis powered by a renewable power source, or both.

14. An apparatus for continuous gas fermentation comprising:
a. a bioreactor comprising a bioreactor inlet, at least one recycle inlet, and a fermentation broth outlet;
b. a separator in fluid communication with the fermentation broth outlet, the separator comprising a liquid outlet and a gas outlet;
c. a vacuum distillation unit and a membrane separator in fluid communication, serially or in parallel, with the separator liquid outlet;
d. the vacuum distillation unit comprising an overhead outlet and a bottoms outlet; and
e. the membrane separator comprising a permeate outlet and a retentate outlet, the retentate outlet in fluid communication with at least one of the bioreactor recycle inlets.

15. The apparatus of claim 14 wherein the bottoms outlet is further in fluid communication with at least one of the bioreactor recycle inlets.

16. The apparatus of claim 14 further comprising a wastewater treatment unit in fluid communication with the permeate outlet and at least one of the bioreactor recycle inlets.

17. The apparatus of claim 14 further comprising a gas treatment unit in fluid communication with the bioreactor inlet.

18. The apparatus of claim 14 further comprising a gas treatment unit in fluid communication with both the gas outlet and at least one of the bioreactor recycle inlets.

19. The apparatus of claim 14 further comprising, in fluid communication the bioreactor inlet, a CO₂ to CO conversion unit selected from a reverse water gas reaction unit, a CO₂ electrolysis unit, a thermo-catalytic conversion unit, an electro-catalytic conversion unit, a partial combustion unit, a plasma conversion unit or any combination thereof.

20. The apparatus of claim 14 further comprising a bleed conduit in fluid communication with the liquid outlet, retentate outlet, or both.
